(19) United States
(12) Patent Application Publication
(10) Pub. No.: US 2006/0141032 A1
(43) Pub. Date: Jun. 29, 2006

(54) Effervescent formulations comprising apomorphine

(76) Inventor: Finn Larsen, Edinburgh (DK)

Correspondence Address: Edwin V. Merkel,;Nixon Peabody, Clinton Square, P.O Box 31051, Rochester, NY, 14603-1051, US

(21) Appl. No.: 10/527,924

(22) PCT Filed: Sep. 19, 2003
(86) PCT No.: PCT/GB03/04141

§ 371 Date: Aug. 10, 2005

(30) Foreign Application Priority Data
Sep. 19, 2002 (GB) 0221711.5

(51) Int. Cl. A61K 9/46

(52) U.S. Cl. 424/466

(57) ABSTRACT

An effervescent formulation comprising apomorphine, preferably comprising multilayer effervescent microspheres containing an acidic substance, a basic substance and water-soluble isolating agent. An effervescent formulation comprising apomorphine wherein dissolution in water of the multilayer effervescent microspheres leads, after almost immediate effervescence, to a solution or a homogeneous dispersion of the apomorphine. The formulation is used for the treatment of male and female sexual dysfunction.

This invention relates to formulations of apomorphine and their use in the treatment of male or female sexual dysfunction.

Sexual dysfunctions are highly prevalent, affecting about 43% of women and 31% of men. Hypoactive sexual desire disorder has been reported in approximately 30% of women and 15% of men in population-based studies, and is associated with a wide variety of medical and psychologic causes. Sexual arousal disorders are found in 10% to 20% of men and women, and is strongly age-related in men. Orgasmic disorder is relatively common in women, affecting about 10% to 15% in community-based studies. In contrast, premature ejaculation is the most common sexual complaint of men, with a reporting rate of approximately 30% in most studies. Finally, sexual pain disorders have been reported in 10% to 15% of women and less than 5% of men. In addition to their widespread prevalence, sexual dysfunctions have been found to impact significantly on interpersonal functioning and overall quality of life in both men and women (Rosen, 2000, Curr Psychiatry Rep, 2, 189-195).

Erectile dysfunction occurs in 10% to 20% of men. It is defined as the inability to achieve and sustain an erection sufficient for intercourse. In any given case this can result from psychological disturbances (psychogenic), from physiological abnormalities in general (organic), from neurological disturbances (neurogenic), hormonal deficiencies (endocrine) or from a combination of the foregoing. Psychogenic factors for erectile dysfunction include such processes as depression, anxiety, and relationship problems which can impair erectile functioning by reducing erotic focus or otherwise reducing awareness of sensory experience. This may lead to an inability to initiate or maintain an erection. Psychotherapy and/or behavioural therapy are often useful for some patients with psychogenic erectile dysfunction.

In the female, sexual dysfunction can arise from organic or psychogenic causes or from a combination of the foregoing. Female sexual dysfunction includes a failure to attain or maintain vaginal lubrication-swelling responses of sexual excitement until completion of the sexual activity. Organic female sexual dysfunction is known to be related in part to vasculogenic impairment resulting in inadequate blood flow, vaginal engorgement insufficiency and clitoral erection insufficiency.

A number of methods for the treatment of male and female sexual dysfunction have been suggested. Pharmacological agents which have been used in the treatment of male erectile dysfunction include orally administered agents such as yohimbine, bromocriptine, fluoxetine, trazadone, trental, sildenafil, phentolamine, and extracts of Ginkgo biloba.

With female sexual dysfunction, a recent study has suggested that sildenafil appears to significantly improve both subjective and physiologic parameters or the female sexual response (Berman et al., 2001, J Sex Marital Ther, 27, 411-420). Also, a recent small pilot study was conducted on the effects of oral phentolamine in menopausal women with female sexual arousal disorder. The study found a mild positive effect of phentolamine across all measures of arousal (Rosen et al., 1999, J Sex Marital Ther, 25, 137-144).

Recently, U.S. Pat. No. 5,624,677 showed that oral administration of apomorphine can be used to induce an erection in a psychogenic male patient, and is suitable for treatment since an apomorphine dose required to achieve a significant erectile response which is not accompanied by nausea and vomiting or other serious undesirable side effects such as arterial hypotension, flushing and diaphoresis is possible. The specific mechanisms by which apomorphine acts to produce an erectile response in a human patient are not yet completely understood but believed to be centrally acting through dopamine receptor stimulation in the medial preoptic area of the brain.

Apomorphine, a derivative of morphine, has been classified as a selective dopamine receptor agonist that stimulates the central nervous system. It has been shown to have very poor oral bioavailability; see, for example, Baldessarini et al in Gessa et al (eds.), Apomorphine and other Dopaminiomimetics, Basic Pharmacology, 219-228, Raven Press, N.Y. (1981). A number of reports describe attempts to identify a suitable means to supply apomorphine for the treatment of male and female sexual dysfunction.

WO 98/31368 discusses a treatment for psychogenic erectile dysfunction using a dopamine agonist such as apomorphine in a form designed to release the active ingredient rapidly in the oral cavity.

U.S. Pat. No. 5,770,606 discusses a treatment for psychogenic erectile dysfunction by a sublingual administration of apomorphine dosage forms so as to maintain a plasma concentration of apomorphine of no more than about 5.5 nanograms per milliliter.

WO 99/66916 suggests that, for optimal erectile response, steady state circulating serum and mid-brain tissue levels of apomorphine are to be maintained within a relatively closely defined range. It also states that the nausea side effect associated with the use of apomorphine can be substantially reduced by administration of an antiemetic agent.

U.S. Pat. No. 5,945,117 discusses the treatment of female sexual dysfunction without substantial undesirable side effects by sublingual administration of apomorphine at a plasma concentration of no more than 5.5 nanograms per milliliter.

WO 00/76509 and WO 02/24202 both suggests apomorphine can be used to treat male and female sexual dysfunction using a nasal delivery system without causing substantial intolerable side effects.

However, there remains a need for alternative methods of supplying a therapeutically appropriate quantity of apomorphine for the treatment of male and female sexual dysfunction. In some cases it may not be appropriate to administer the apomorphine using the prior disclosed method, for example, in instances where the patient has difficulty in swallowing tablets or nasally absorbing the treatment.

A first aspect of the invention provides an effervescent formulation comprising apomorphine. It may be used for the treatment of male or female sexual dysfunction. Such a formulation, when dissolved in water, typically leads to a homogenous dispersal of the apomorphine.

By ‘effervescent formulation’ we mean that the formulation is effervescent when placed in an aqueous solution.

By ‘apomorphine’ we include free base apomorphine or a pharmaceutically acceptable salt of apomorphine. Suitable salts include the hydrochloride, the hydrobromide, the hydroiodide, the bisulphate, the phosphate, the acid phosphate, the lactate, the citrate, the tartrate, the salicylate, the succinate, the maleate, the gluconate, the acetate, the trifluoroacetate, and the like. It is preferred that the apomorphine is in the form of the hydrochloride salt.

Particularly preferred is apomorphine hydrochloride: (6aR)-5,6,6a,7-Tetrahydro-6-methyl-4H-dibenzo[de,g]quinoline-10,11-diol hydrochloride hemihydrate.

Effervescent formulations offer an advantage over the existing forms of supplying apomorphine as they have a high level of patient acceptability. The formulation may be placed on the tongue where they effervesce, and release the apomorphine.

A preferred embodiment of the invention is that the effervescent formulation comprises multilayer effervescent microspheres. The manufacture of certain suitable multilayer effervescent microspheres is described in WO 98/31342 and U.S. Pat. No. 6,210,711 B1, hereby incorporated by reference in their entirety.

A still further embodiment of the invention is that the multilayer effervescent microspheres contain an acidic substance, a basic substance and water-soluble isolating agent.

The term ‘microsphere’ will be intended to refer to microgranules formed of a support material consisting of a matrix in which the apomorphine is dispersed. In accordance with the European Pharmacopoeia monograph on spheres, microspheres have an average diameter of less than 1.0 mm and greater than or equal to 1.0 μm. They are generally intended for oral or parenteral administration and are used either as constituents of pharmaceutical form, such as tablets, or in their natural form combined or otherwise with other excipients, and distributed or otherwise in unit doses, such as sachets, gel-capsules or powder for injectable preparation.

The ‘water-soluble isolating agent’ may be any such agent which serves as both a binder and as an isolating barrier intended to avoid an effervescent reaction between the alkaline substance and the acidic substance during the preparation process but also during storage of the microspheres, irrespective of the storage conditions. Typically, it is chosen from polyvinylpyrrolidone, hydroxypropyl cellulose, methyl cellulose, lactose and sucrose.

By ‘acidic substance’ we include a powder of acidic nature containing an organic acid, for example citric acid, ascorbic acid or acetylleucine.

By ‘basic substance’ we mean a powder of alkaline nature containing a sodium bicarbonate or any other carbonate usually used in the preparation of effervescent forms, such as lithium hydrogen carbonate, monosodium carbonate, lithium glycine carbonate, monopotassium carbonate, calcium carbonate or magnesium carbonate. It is preferred that the ‘basic substance’ is a sodium salt such as sodium bicarbonate.

A preferred embodiment of the invention relates to multilayer effervescent microspheres containing an acidic substance, a basic substance and a water-soluble isolating agent whose dissolution in water leads, after almost immediate effervescence, to a solution or a homogeneous dispersion of apomorphine.

According to a first variant of this embodiment of the invention, the water-soluble isolating agent is dispersed in the entire bulk of each microsphere, the latter having a two-layer structure: a layer of acidic substance in which is dispersed the water-soluble isolating agent and a layer of alkaline substance in which is dispersed the water-soluble isolating agent.

According to a second variant of this embodiment of the invention, the water-soluble isolating agent is in the form of a thin film separating the acidic and alkaline substances. In this case, each microsphere has a three-layer structure: a layer of acidic substance and a layer of alkaline substance separated by a layer of water-soluble isolating agent.

Whether the microspheres have a two-layer or three-layer structure, the water-soluble isolating agent serves two purposes; it acts as a binder and as an isolating barrier intended to avoid an effervescence reaction between the alkaline substance and the acidic substance during the preparation process but also during storage of the microspheres, irrespective of the storage conditions.

In a preferred embodiment of the invention the effervescent formulation contains apomorphine present in a unit dose amount of from about 0.5 mg to 50 mg such as 0.5 mg, 1 mg, 1.5 mg, 2 mg, 2.5 mg, 3 mg, 3.5 mg, 4 mg, 4.5 mg 5 mg, 10 mg, 20 mg, 30 mg, 40 mg or 50 mg. Most preferably the apomorphine is present in a unit dose amount of 2 mg to 3 mg.

In a further embodiment the effervescent formulation of the invention is presented in a tablet form. Methods of forming tablets suitable for the invention from such an effervescent formulation are well known to those skilled in the art. A tablet may be made by compression or moulding, optionally with one or more accessory ingredients. Compressed tablets may be prepared by compressing in a suitable machine the active ingredient in a free-flowing form such as a powder or granules. Moulded tablets may be made by moulding in a suitable machine a mixture of the powdered compound moistened with an inert liquid diluent.

In a further embodiment the effervescent formulation of the invention is presented in a powder form. Methods of forming powders suitable for the invention from such an effervescent formulation are well known to those skilled in the art.

It is preferred that when the formulation contains microspheres, the apomorphine is not present within the microspheres. For example, when microspheres are tabletted to form a tablet the apomorphine is preferably present on or between the microspheres in the tablet.

The apomorphine may, however, in some embodiments, be present in the microspheres.

A further aspect of the invention is a process for making an effervescent formulation containing apomorphine.

A preferred embodiment of the invention is a process wherein the effervescent formulation comprises multilayer effervescent microspheres containing an acidic substance, a basic substance, and a water-soluble isolating agent which upon dissolution in water leads, after almost immediate effervescence, to a solution or a homogeneous dispersion of apomorphine.

In a preferred embodiment of the invention, the apomorphine is not present within the microspheres.

In a further embodiment of the process of the invention the acidic and/or basic substances contains or contain apomorphine.

In a further preferred embodiment of the process of the invention the process employs the method of rotary granulation in a fluidized air bed.

The advantage of rotary granulation applied to these effervescent compositions is the continuous linking of the operations in one and the same chamber which, as a result of the components used and certain precautions taken, induces no effervescence. Furthermore, this rotary granulation technique allows the relative proportions of the various compounds to be modified, in particular the relative molar proportions of the acidic and basic fractions.

Specifically, the process according to the invention makes it possible advantageously to obtain effervescent forms whose relative proportion of alkaline and acidic fractions is less than the stoichiometric proportion implemented in the prior art for effervescent tablets manufactured by the granulation method, without the quality of the effervescence being adversely affected.

In particular, the relative proportion of the basic and acidic substances implemented in the context of the process according to the invention is less than 0.6, in particular less than 0.25.

All the steps of the process according to the invention are carried out under atmospheric pressure, without any specific dehydration system or any specific precautions.

The apparatus used to carry out the process for preparing the effervescent microspheres is, for example, apparatus constructed by the company Glatt, onto which a rotor tank is fitted.

Such an item of apparatus is described in patent EP 0,505,319, which we include, by way of reference, in the present application.

Also subject of the present invention is, firstly, a process for preparing effervescent microspheres which have a two-layer structure according to the first variant described above.

Said process is performed by rotary granulation in a fluidized air bed combined with a system for spraying powder and a system for the tangential spraying of wetting liquid. The process comprises two continuous steps, a first step of spheronization of microspheres using a powder A and a second step of spheronization of a powder B on the microspheres of powder A, one of the powders A and B being acidic and the other alkaline and it being possible for each of them to contain or consist of apomorphine. It is is preferred that powders A or B contain but do not consist of apomorphine.

During the first spheronization, the powder A is placed in the moving rotary granulation tank and suspended in the air bed. The components of the powder A are mixed together for five minutes and the air inlet temperature is stabilised to a temperature T₀.

The powder A thus blended is sprayed with a wetting liquid containing the water-soluble isolating agent. The microspheres of powder A obtained are dried by bringing the air inlet temperature to Ts and are then optionally screened using a 1000 μm screen. During the second spheronization, the air inlet temperature is brought to T₀. The powder B and the wetting liquid containing the water-soluble isolating agent are then simultaneously sprayed onto the dried powder A microspheres obtained previously. The powder B is sprayed by means of the powder spraying system installed on the Glatt apparatus. The two-layer microspheres obtained are dried by bringing the air inlet temperature to Ts. After drying, the microspheres must be packaged quickly, but a small amount of moisture uptake does not harm the storage.

During the two spheronizations, the wetting liquid containing the water-soluble isolating agent is the same, for example polyvinylpyrrolidone (PVP) dissolved in an alcohol or an aqueous-alcoholic mixture, in particular PVP dissolved to 4% by weight in ethanol at 60% by volume.

The two-layer microspheres obtained according to the process of the invention have an average particle size of between 20 and 500 μm.

A subject of the present invention is also a process for preparing effervescent microspheres which have a three-layer structure according to the second variant described above.

Said process is performed according to the method of rotary granulation in a fluidized air bed combined with a system for the tangential spraying of wetting liquid.

The process comprises three continuous steps, a first step of spheronization of microspheres using a powder A, a second step of spheronization of a water-soluble isolating agent on the microspheres of powder A, and then a third step of spheronization of a powder B on the microspheres A protected with a film of water-soluble isolating agent, one of the powders A and B being acidic and the other alkaline and it being possible for each of them to contain or consist of apomorphine. It is preferred that powders A or B contain but do not consist of apomorphine.

During the first spheronization, the powder A containing an added binder, for example PVP, is placed in the moving tank and suspended in the air bed. The components of the powder A are mixed together for five minutes and the air inlet temperature is stabilized to T₀. The powder A thus blended is sprayed with a wetting liquid. The microspheres of powder A obtained are dried by bringing the air inlet temperature to Ts. During the second spheronization, the air inlet temperature is brought to T₀. The water-soluble isolating agent is added directly to the tank and the wetting liquid sprayed until microspheres of powder A which are coated with a film of water-soluble isolating agent are obtained, and are dried by bringing the air inlet temperature to Ts. After drying, the coated microspheres are screened and the powder B is then added directly to the rotary granulation tank when the air inlet temperature has stabilized at T₀. The three-layer microspheres are obtained by spraying the preceding microspheres with a wetting liquid. The three-layer microspheres obtained are dried by bringing the air inlet temperature to Ts. After drying, the microspheres must be packaged quickly, but a small amount of moisture uptake does not harm the storage.

During the first two steps, the wetting liquid is, for example, an aqueous-alcoholic solution, in particular ethanol at 60% by volume. During the final step, the water-soluble isolating agent can be introduced by means of the powder B, in which case the wetting liquid used will be the same as during the first two steps, or alternatively the isolating agent is introduced by means of the wetting liquid, which will be an alcoholic or aqueous-alcoholic solution containing the isolating agent, for example PVP dissolved to 4% by weight in ethanol at 60% by volume.

The three-layer microspheres obtained according to the process of the invention have an average particle size of between 200 and 1000 μm.

According to the process for manufacturing microspheres, whether they are two-layer or three-layer microspheres, the powder of alkaline nature contains a sodium bicarbonate or any other carbonate usually used in the preparation of effervescent forms, such as lithium hydrogen carbonate, monosodium carbonate, lithium glycine carbonate, monopotassium carbonate, calcium carbonate, magnesium carbonate and, optionally apomorphine; whereas the powder of acidic nature contains an organic acid, for example citric acid, ascorbic acid, acetylleucine and, optionally, apomorphine. It is preferred that the apomorphine is not present within the microspheres, but rather is present on or between them in the final formulation (typically a tablet). In some embodiments, however, the powder of alkaline nature and the powder of acidic nature contain but do not consist of apomorphine.

In a further embodiment of the process of the invention the acidic and alkaline powders can also contain a diluent, for example lactose or Glucidex; flavorings and sweeteners, for example orange flavoring, citric acid, sodium saccharinate; various excipients.

In a preferred embodiment of the process of the invention apomorphine is present such that the resulting effervescent formulation contains apomorphine present in a unit dose amount of from between 0.5 mg and 50 mg, typically 0.5 mg, 1 mg, 1.5 mg, 2 mg, 2.5 mg, 3 mg, 3.5 mg, 4 mg, 4.5 mg 5 mg, 10 mg, 20 mg, 30 mg, 40 mg or 50 mg. Most preferably the process produces a formulation where apomorphine is present in a unit dose amount of 2 mg to 3 mg.

In a further embodiment of the process of the invention the effervescent formulation of the invention is presented in a tablet form. Methods of forming tablets suitable for the invention from such an effervescent formulation are well known to those skilled in the art as described above. Preferably, the apomorphine is present in the tablet on or between microspheres (when present).

According to one embodiment of the invention, the powder A is of alkaline nature and the powder B is of acidic nature.

According to another embodiment of the invention, the powder B is of alkaline nature and the powder A of acidic nature.

The wetting liquid is sprayed by means of a nozzle 1.2 mm in diameter, at an average flow rate of between 10 and 30 g/min. The air inlet temperature of the fluidized bed is between 55 and 65° C. during the spheronization steps (T₀) and between 75 and 85° C. during the drying phases (Ts).

The microspheres obtained according to the process of the invention contain 5 to 75% of alkaline substance, 10 to 75% of acidic substance, 3 to 15% of water-soluble isolating agent, 5 to 50% of diluent and 1 to 30% of flavorings and sweeteners and an appropriate amount of apomorphine, for example 0.2% to 4% apomorphine.

The relative humidity of the microspheres obtained according to the process of the invention, measured for fifteen minutes by the infrared balance method at 90° C., is between 1 and 2% at the rotary granulation tank outlet.

The overall yield for the process is calculated from the fraction of particles smaller than 2500 μm in size, the working yield of the spheres corresponds to the fraction of particles between 200 and 1000 μm, for the process for preparing three-layer microspheres, between 20 and 500 μm for the process for preparing two-layer microspheres.

The feasibility of the process according to the invention is evaluated according to the ease with which the microspheres are obtained, the speed of production of a batch and the yield for each step.

Analysis of the batches includes particle size analysis of a sample of 100 g of spheres by the superimposed screens method (sample obtained from the total fraction of a batch), after which a morphological study of the microspheres obtained, relating to the overall appearance, sphericity, cohesion and uniformity of the particles, is carried out by examination with a binocular magnifying glass.

According to one variant of the invention, the two-layer or three-layer effervescent microspheres are manufactured by the mounting technique combined with a system for the tangential spraying of wetting liquid. The powder A and the powder B can be mounted successively on spheres containing apomorphine coated with water-soluble isolating agent, or on neutral spheres.

A further aspect of the invention is an effervescent formulation of apomorphine obtained or obtainable by any one of the processes of the invention mentioned above.

A further aspect of the invention provides an effervescent formulation of apomorphine for use in medicine

A further aspect of the invention provides a pharmaceutical composition which comprises an effervescent formulation of apomorphine according to the invention and a pharmaceutically acceptable carrier.

A further aspect of the invention is a method of treating human male or female sexual dysfunction comprising administering to said human an effervescent formulation of apomorphine according to the invention and/or obtained or obtainable by a process according to the invention.

A further aspect of the invention is the use of an effervescent formulation of apomorphine according to the invention and/or obtained or obtainable by a process according to the invention in the manufacture of a medicament for the treatment of male or female sexual dysfunction.

Preferred embodiments of the invention are described in the following processes.

Process 1: Process for preparing multilayer effervescent microspheres containing an acidic substance, a basic substance, and a water-soluble isolating agent which upon dissolution in water leads, after almost immediate effervescence, to a solution or a homogeneous dispersion of apomorphine, wherein the acidic and basic substances contain or consist of apomorphine, which employs the method of rotary granulation in a fluidized air bed.

Process 2. Process for preparing microspheres defined in process 1, which employs the method of rotary granulation in a fluidized air bed combined with a system for spraying powder and a system for the tangential spraying of wetting liquid, which comprises two continuous steps, a first step of spheronization of microspheres using a powder A and a second step of spheronization of a powder B on the microspheres of powder A, one of the powders A and B being acidic and the other alkaline.

Process 3. Process according to process 2, wherein the powder A is introduced directly into the rotary granulation tank and then sprayed with a wetting liquid containing the water-soluble isolating agent, while the powder B and a wetting liquid containing the water-soluble isolating agent are simultaneously and respectively sprayed via the system for spraying powder and the system for the tangential spraying of liquid.

Process 4. Process according to process 3, wherein the microspheres obtained have an average particle size of between 20 and 500 μm.

Process 5. Process for preparing microspheres as defined in process 1, which employs the method of rotary granulation in a fluidized air bed combined with a system for the tangential spraying of wetting liquid, which comprises three continuous steps, a first step of spheronization of microspheres using a powder A, a second step of spheronization of a water-soluble isolating agent on the microspheres of powder A, and then a third step of spheronization of a powder B on the microspheres A protected with a film of water-soluble isolating agent, one of the powders A and B being acidic and the other alkaline.

Process 6. Process according to process 5, wherein the powder A and the water-soluble isolating agent are sprayed with an alcoholic or aqueous-alcoholic solution.

Process 7. Process according to process 5, wherein the powder B contains the water-soluble isolating agent and is sprayed with an alcoholic or aqueous-alcoholic solution.

Process 8. Process according to process 5, wherein the powder B is sprayed with a wetting liquid containing the water-soluble isolating agent.

Process 9. Process according to process 5, wherein the microspheres obtained have an average particle size of between 200 and 1000 μm.

Process 10. Process according to process 3, wherein the wetting liquid containing the water-soluble isolating agent is polyvinylpyrrolidone dissolved in an alcohol or an aqueous-alcoholic mixture, which is polyvinylpyrrolidone dissolved to 4% by weight in ethanol at 60% by volume.

Process 11. Process according to process 2 or 5, wherein the powder of alkaline nature contains a sodium bicarbonate or another carbonate used in the preparation of effervescent forms, selected from lithium hydrogen carbonate, monosodium carbonate, lithium glycine carbonate, monopotassium carbonate, calcium carbonate, magnesium carbonate; and apomorphine.

Process 12. Process according to process 2 or 5, wherein the powder of acidic nature contains citric acid or ascorbic acid or, acetylleucine, and/or apomorphine.

Process 13. Process according to process 1, wherein the powder of alkaline nature also contain an edible diluent and; flavorings and sweeteners.

Process 14. Process according to process 2 or 5, wherein the microspheres obtained contain 5 to 75% of alkaline substance, 10 to 75% of acidic substance, 3 to 15% of water-soluble isolating agent, 5 to 50% of diluent, and 1 to 30% of flavorings and sweeteners.

Process 15. Process according to process 2 or 5, wherein the powder A is of alkaline nature and the powder B of acidic nature.

Process 16. Process according to process 2 or 5, wherein the powder A is of acidic nature and the powder B of alkaline nature.

Process 17. Process according to process 3 or 6, wherein the wetting liquid is sprayed by means of a nozzle 1.2 mm in diameter, at an average flow rate of between 10 and 30 g/min.

Process 18. Process according to process 2 or 5, wherein the air inlet temperature of the fluidized bed is between 55 and 65° C. during spheronization steps, and between 75 and 85° C. during drying phases associated with the spheronization steps.

Process 19. Process according to process 2 or 5, wherein the relative humidity of the microspheres obtained is between 1 and 2% at the rotary granulation tank outlet.

Process 20. Process for preparing microspheres as defined in process 1, which employs the mounting technique combined with a system for the tangential spraying of wetting liquid.

Process 21. Process according to process 20, wherein the powder A and the powder B are mounted successively on spheres containing apomorphine coated with water-soluble isolating agent, or on neutral spheres.

Process 22. Process according to process 12, wherein the powder of acidic nature also contains an edible diluent and flavorings and sweeteners.

The examples which follow illustrate the invention without limiting its scope.

The percentages are expressed on a weight basis.

### EXAMPLE 1

Two-Layer Effervescent Microspheres Containing Ascorbic Acid (Vitamin C)

Alkaline microspheres are prepared, on which is deposited the acidic substance (vitamin C).

The table below gives the details of the formulation used.

| FORMULATION | COMPONENT | PERCENTAGE |
|---|---|---|
| Powder A | | |
| Alkaline compound | Sodium | 20% |
| | bicarbonate | |
| Diluent | Lactose | 6% |
| Sweetener | Glucidex 6 .RTM. | 6% |
| Powder B | | |
| Acidic compound | Ascorbic acid | 48% |
| Apomorphine hydrochloride | | 2% |
| hemihydrate | | |
| Flavoring | Orange flavoring | 1% |
| Sweeteners | Sodium | 0.3% |
| | saccharinate | |
| | Glucidex 6 .RTM. | 6.35% |
| Diluent | Lactose | 6.35% |

The wetting liquid used during the two successive rotary granulations is an aqueous-alcoholic PVP solution containing 4% PVP in ethanol at 60% by volume.

This mixture is sprayed at an average flow rate of 25 grams per minute.

In this formulation, the lactose is combined in equal part with Glucidex 60, although it is possible to use lactose alone.

The powder formulations A and B are prepared on batches of variable size of 1000 to 5000 g with, depending on the case, use of equipment from the company Glatt.

The effervescent spheres obtained have a fairly uniform appearance and a majority particle size of fractions between 200 and 500 μm. The relative humidity is 1.6% at the rotary granulation tank outlet.

### EXAMPLE 2

Two-Layer Effervescent Microspheres Containing Acetylleucine

Alkaline microspheres are prepared, on which is deposited the acidic substance (acetylleucine) under the same conditions as in Example 1.

The table below gives the details of the formulation used.

| FORMULATION | COMPONENT | PERCENTAGE |
|---|---|---|
| Powder A | | |
| Alkaline compound | Sodium | 20% |
| | bicarbonate | |
| Diluent | Lactose | 9.85% |
| Powder B | | |
| Acidic compound | Acetylleucine | 49% |
| Apomorphine hydrochloride | | 1% |
| hemihydrate | | |
| Flavoring | Orange flavoring | 1% |
| Sweetener | Sodium | 0.3% |
| | saccharinate | |
| Diluent | Lactose | 9.85% |

The particle size distribution of the batch is a majority for the fractions 25 to 500 μm.

The relative humidity is 1.9% at the rotary granulation tank outlet.

According to the size of the batches ranging from 1000 to 10,000 g, apparatus GPCG 1 or GPCG 5 from the company Glatt with a rotor tank mounting [lacuna].

### EXAMPLE 3

Three-Layer Effervescent Microspheres Containing Ascorbic Acid (Vitamin C)

Three-layer effervescent microspheres are manufactured, comprising an alkaline core isolated from the acidic substance, ascorbic acid, by means of a film of PVP.

| FORMULATION | COMPONENT | PERCENTAGE |
|---|---|---|
| Powder A | | |
| Alkaline compound | Sodium | 25% |
| | bicarbonate | |
| Binder | PVP K30 | 1.316% |
| Diluent | Lactose | 7.950% |
| Water-soluble | PVP K30 | 6.958% |
| isolating agent | | |
| Powder B | | |
| Acidic compound | Ascorbic acid | 46% |
| Apomorphine hydrochloride | | 4% |
| hemihydrate | | |
| Flavoring | Orange flavoring | 1% |
| Sweeteners | Sodium | 0.2% |
| | saccharinate | |
| | Citric acid | 1% |
| Diluent | Lactose | 6.950% |

The test is carried out in apparatus of GPCG1 type from the company Glatt, with the rotor tank mounting.

1460 g of ethanol at 60% by volume are sprayed in total during the three steps, at an average flow rate of 15 grams per minute.

The size of the final batch is 1000 g.

The working yield corresponding to the fraction of particles between 200 and 1000 μm is 65%. The relative humidity is 1.5% at the tank outlet.

### EXAMPLE 4

### Preparation of Effervescent Tablets Containing Apomorphine

Effervescent tablets containing 2 mg of apomorphine chlorhydrate (ie apomorphine hydrochloride) were prepared so that the time for drug dissolution or tablet disintegration and/or dissolution is less than 10 minutes.

In the laboratory scale manufacturing process, apomorphine has been mixed with effervescent microspheres prepared as described above with a Glatt GPCGI. The mixture was then added with diluent (mannitol), lubricants (magnesium stearate, talc), flavouring and tabletted on a single punch alternative press under isolator.

In the industrial scale process, apomorphine is introduced directly on the effervescent microspheres directly in the Glatt by the powder device. After drying, the spheres are mixed with the other excipients and tabletted.

The compatibility between apomorphine and the excipients needed to produce the effervescent microspheres was tested. This was done by mixing 2 by 2 every excipient with apomorphine and placing the samples at room temperature or on a store at 30° C. and 40° C. and looking at one and three months the aspect, colour, titre of apomorphine and any degradation products. In addition, at the same time, the stability of a non-formal formulation has been followed on the taste and dissolution/disintegration time over a four month period and any modification detected.

The formulations show good stability for apomorphine content and dissolution rate at 25° C. and 40° C., except some slight colour at 40° C. without any modification of the apomorphine content or any appearance of degradation product.

The following tables show the composition of the tablets (mannitol is used for increasing the dissolution time) and the results of stability studies at three months.

RESULTS OF STABILITY OF APOMORPHINE FORMULATIONS (I)
BATCH NO: F17S048
DOSE: 2 mg
PACKAGING: Bottle brown glass
BATCH SIZE: about 1500 units

| | RESULTS | | | | | |
|---|---|---|---|---|---|---|
| | | T1 month | T1 month | T3 months | T3 months | PROVISIONAL |
| ASSAYS | T0 | 25° C. | 40° C. | 25° C. | 40° C. | STANDARDS |
| General characteristics | | | | | | |
| Organoleptic characteristics | Conform | Conform | Conform | Conform | Slightly | White tablets, |
| | | | | | green | hemispheric |
| Thickness | 2.88 mm | 3.07 mm | 2.99 mm | 3.19 mm | 2.95 mm | — |
| Tablets hardness | 65.5 N | 65.1 N | 69.0 N | 58.2 N | 67.5 N | about 60 N |
| Friability | 0.80% | NR | NR | 0.71% | 0.49% | <1% |
| Residual humidity | −0.98% | −0.97% | −2.00% | −1.02% | −0.99% | |
| Effervescence time | 34 sec | 37 sec | 41 sec | 41 sec | 42 sec | <1 minute |
| Mass mean | 97.9 mg | 100.0 mg | 96.0 mg | 98.3 mg | 93.0 mg | 100 mg ± 7.5% |
| | | | | | | (from 92.5 to 107.5 mg) |
| | | | | | | Mass mean (cFEP) |
| Drug assay | | | | | | |
| (HPLC) | | | | | | |
| Unit/per | 2.00 mg | 1.91 mg | 1.93 mg | 2.01 mg | 1.83 mg | 2 mg/tablet ± 5% |
| | (v = 9.1%) | (v = 17.1%) | (v = 17.6%) | (v = 10.5%) | (v = 24.4%) | (from 1.9 mg to 2.1 mg) |
| According to tablet weight | 2.00 mg | 1.93 mg | 2.01 mg | 1.99 mg | 1.98 mg | |
| | (v = 3.2%) | (v = 11.2%) | (v = 12.1%) | (v = 6.6%) | (v = 15.5%) | |
| Related substances | | | | | | |
| (HPLC) | | | | | | |
| European Pharmacopeia | Conform | Conform | Conform | Conform | Conform | all impurities = max |
| | (total = 0.15%) | (total = 0.22%) | (total = 0.17%) | (total = 0.17%) | (total = 0.09%) | 0.2% |
| | | | | | | total impurities = mix |
| | | | | | | 0.8% |
| % additional peaks | 0.15% | 0.13% | 0.14% | 0.05% | 0% | — |

RESULTS OF STABILITY OF APOMORPHINE FORMULATIONS (II)
BATCH NO: F18S049
DOSE: 2 mg
PACKAGING: Bottle brown glass
BATCH SIZE: about 1500 units

| | RESULTS | | | | | |
|---|---|---|---|---|---|---|
| | | T1 month | T1 month | T3 months | T3 months | PROVISIONAL |
| ASSAYS | T0 | 25° C. | 40° C. | 25° C. | 40° C. | STANDARDS |
| General characteristics | | | | | | |
| Organoleptic characteristics | Conform | Conform | Conform | Conform | Slightly green | White tablets, |
| | | | | | | hemispheric |
| Thickness | 3.96 mm | 3.95 mm | 4.02 mm | 3.99 mm | 4.06 mm | — |
| Tablets hardness | 83.0 N | 85.7 N | 96.1 N | 77.9 N | 93.7 N | about 80 N |
| Friability | 0.57% | NR | NR | 0.60% | 0.38% | <1% |
| Residual humidity | −0.42% | −0.50% | −0.80% | −0.49% | −0.80% | |
| Effervescence time | 2 min 09 | 1 min 59 | 2 min 01 | 2 min 01 | 2 min | 3 minutes |
| Mass mean | 238.7 mg | 240.4 mg | 239.5 mg | 242.5 mg | 237.8 mg | 250 mg ± 5% |
| | | | | | | (from 237.5 to 262.5 mg) |
| | | | | | | Mass mean (cFEP) |
| Drug assay | | | | | | |
| (HPLC) | | | | | | |
| Unit/per | 1.93 mg | 1.95 mg | 1.93 mg | 2.03 mg | 1.94 mg | 2 mg/tablet ± 5% |
| | (v = 3.7%) | (v = 2.2%) | (v = 7.1%) | (v = 2.4%) | (v = 5.3%) | (from 1.9 mg to 2.1 mg) |
| According to tablet weight | 2.01 mg | 2.04 mg | 2.03 mg | 2.08 mg | 2.03 mg | |
| | (v = 2.3%) | (v = 3.5% | (v = 5.5%) | (v = 3.3%) | (v = 4.5%) | |
| Related substances | | | | | | |
| (HPLC) | | | | | | |
| European Pharmacopeia | Conform | Conform | Conform | Conform | Conform | all impurities = max |
| | (total = 0.35%) | (total = 0.36% | (total = 0.31%) | (total = 0.10%) | (total = 0.11%) | 0.2% |
| | | | | | | total impurities = mix |
| | | | | | | 0.8% |
| % additional peaks | 0.26% | 0.20% | 0.23% | 0% | 0% | — |

RESULTS OF STABILITY OF APOMORPHINE FORMULATIONS (III)
BATCH NO: F19S050
DOSE: 2 mg
PACKAGING: Bottle brown glass
BATCH SIZE: about 1200 units

| | RESULTS | | | | | |
|---|---|---|---|---|---|---|
| | | T1 month | T1 month | T3 months | T3 months | PROVISIONAL |
| ASSAYS | T0 | 25° C. | 40° C. | 25° C. | 40° C. | STANDARDS |
| General characteristics | | | | | | |
| Organoleptic characteristics | Conform | Conform | Conform | Conform | Slightly green | White tablets, |
| | | | | | | hemispheric |
| Thickness | 4.13 mm | 4.16 mm | 4.16 mm | 4.15 mm | 4.14 mm | — |
| Tablets hardness | 82.8 N | 77.2 N | 107.1 N | 88.3 N | 88.5 N | about 80 N |
| Friability | 0.80% | NR | NR | 0.53% | 0.25% | <1% |
| Residual humidity | −2.88% | −3.10% | −4.12% | −3.81% | −4.82% | |
| | | (tablets burst) | (tablets burst) | (tablets burst) | (tablets burst) | |
| Effervescence time | 7 min 56 | 8 min 04 | 8 min 01 | 8 min 16 | 8 min 07 | 8 minutes |
| Mass mean | 242.9 mg | 243.3 mg | 244.6 mg | 246.5 mg | 245.4 mg | 250 mg ± 5% |
| | | | | | | (from 237.5 to 262.5 mg) |
| | | | | | | Mass mean ± 5% |
| | | | | | | (cFEP) |
| Drug assay | | | | | | |
| (HPLC) | | | | | | |
| Unit/per | 2.00 mg | 2.02 mg | 1.95 mg | 2.01 mg | 2.02 mg | 2 mg/tablet ± 5% |
| | (v = 3.3%) | (v = 5%) | (v = 7.4%) | (v = 3.6%) | (v = 1.7%) | (from 1.9 mg to 2.1 mg) |
| According to tablet weight | 2.02 mg | 2.05 mg | 2.05 mg | 2.05 mg | 2.07 mg | |
| | (v = 2.1%) | (v = 4.1% | (v = 3.4%) | (v = 2.8%) | (v = 2%) | |
| Related substances | | | | | | |
| (HPLC) | | | | | | |
| European Pharmacopeia | Conform | Conform | Conform | Conform | Conform | all impurities = max |
| | (total = 0.29%) | (total = 0.16% | (total = 0.22%) | (total = 0.14%) | (total = 0.19%) | 0.2% |
| | | | | | | total impurities = mix |
| | | | | | | 0.8% |
| % additional peaks | 0.18% | 0.01% | 0.08% | 0% | 0% | — |

Batch composition no. 2022401F17S048
Batch Size: 400 g and about 1500 units
Effervescence time: <1 minute

| Name of the Raw materials | | | | |
|---|---|---|---|---|
| (DCI and commercial | Batch No. | Centesimal | Unit formula | Manufacturing |
| name) | (Supplier) | formula | (mg) | (g) |
| 1. Apomorphine Chlorydrate | 2R00001 FRANCOPIA | 2.00 | 2.00 | 8.00 |
| 2. Acid citric | / | 60.31 | 60.31 | 366.00 |
| | 0/1399308 | | | |
| 3. Sodium Bicarbonate | F09S027 | 25.85 | 25.85 | |
| | SOLVAY | | | |
| 4. Polyvinylpyrrolidone K30 | 47-0090 | 5.34 | 5.34 | |
| Kollidon 30 | BASF | | | |
| 5. Flavour mint EH0159 | ECH 99/22886 | 3.00 | 3.00 | 12.00 |
| E40159 | PHARMAROME | | | |
| 6. Aspartame | G7727/7 | 1.50 | 1.50 | 6.50 |
| | COOPER | | | |
| 7. Magnesium Stearate | S52526/1 | 1.00 | 1.00 | 4.00 |
| | COOPER | | | |
| 8. Talc 00 | G8028/1 | 1.00 | 1.00 | 4.00 |
| | COOPER | | | |
| | TOTAL | 100.00 | 100.00 | 400.00 |

Galenical properties

| | |
|---|---|
| 1 | Active ingredient |
| 2 | Acid effervescent agent |
| 3 | Basic effervescent agent |
| 4 | Binder |
| 5 | Flavour |
| 6 | Sweetening substance |
| 7 | Lubricant |
| 8 | Lubricant |
| 9 | |
| 10 | |

Batch composition no. 2022401F17S049
Batch Size: 750 g and about 1500 units
Effervescence time: about 2 minutes

| Name of the Raw materials | | | | |
|---|---|---|---|---|
| (DCI and commercial | Batch No. | | | Manufacturing |
| name) | (Supplier) | Centesimal formula | Unit formula (mg) | (g) |
| 1. Apomorphine Chlorydrate | 2R00001 FRANCOPIA | 0.80 | 2.00 | 6.00 |
| 2. Acid citric | / | 30.55 | 76.37 | 347.62 |
| | 0/1399308 | | | |
| 3. Sodium Bicarbonate | F09S027 | 13.09 | 32.73 | |
| | SOLVAY | | | |
| 4. Polyvinylpyrrolidone K30 | 47-0090 | 2.71 | 6.77 | |
| Kollidon 30 | BASF | | | |
| 5. Flavour mint EH0159 | ECH 99/22886 | 3.00 | 7.50 | 23.50 |
| E40159 | PHARMAROME | | | |
| 6. Aspartame | G7727/7 | 1.50 | 3.75 | 11.25 |
| | COOPER | | | |
| 7. Magnesium Stearate | S52526/1 | 1.00 | 2.50 | 7.50 |
| | COOPER | | | |
| 8. Talc 00 | G8028/1 | 1.00 | 2.50 | 7.50 |
| | COOPER | | | |
| 9. Mannitol 60 | E028L | 46.35 | 115.88 | 347.63 |
| | ROQUETTE | | | |
| | TOTAL | 100.00 | 250.00 | 750.00 |

Galenical properties

| | |
|---|---|
| 1 | Active ingredient |
| 2 | Acid effervescent agent |
| 3 | Basic effervescent agent |
| 4 | Binder |
| 5 | Flavour |
| 6 | Sweetening substance |
| 7 | Lubricant |
| 8 | Lubricant |
| 9 | Thinner |
| 10 | |

Batch composition no. 2022401F17S050
Batch Size: 750 g and about 1200 units
Effervescence time: about 8 minutes

| Name of the Raw materials | | | | |
|---|---|---|---|---|
| (DCI and commercial | Batch No. | | | Manufacturing |
| name) | (Supplier) | Centesimal formula | Unit formula (mg) | (g) |
| 1. Apomorphine Chlorydrate | 2R00001 FRANCOPIA | 0.80 | 2.00 | 6.00 |
| 2. Acid citric | / | 11.49 | 28.72 | 130.81 |
| 3. Sodium Bicarbonate | 0/1399308 | 4.93 | 12.33 | |
| | SOLVAY | | | |
| 4. Kollidon 30 | 47-0090 | 1.02 | 2.55 | |
| | BASF | | | |
| 5. Mannitol 60 | E028L | 37.63 | 94.07 | 282.22 |
| | ROQUETTE | | | |
| 6. Kleptose | E0300 | 37.63 | 94.08 | 282.22 |
| | ROQUETTE | | | |
| 7. Orange flavour grapefruit | PHARMAROME | 3.00 | 7.50 | 22.50 |
| 8. Aspartam | G7727/7 | 1.50 | 3.75 | 11.25 |
| | COOPER | | | |
| 9. Magnesium stearate | S52526/1 | 1.00 | 2.50 | 7.50 |
| 10. Talc 00 | G8028/1 | 1.00 | 2.50 | 7.50 |
| | COOPER | | | |
| | TOTAL | 100.00 | 250.00 | 750.00 |

Galenical properties

| | |
|---|---|
| 1 | Active ingredient |
| 2 | Acid effervescent agent |
| 3 | Basic effervescent agent |
| 4 | Binder |
| 5 | Thinner |
| 6 | Thinner |
| 7 | Flavour |
| 8 | Sweetening substance |
| 9 | Lubricant |
| 10 | Lubricant |

### EXAMPLE 5

### Treatment of Psychogenic Erectile Dysfunction with Apomorphine.

A male patient presenting symptoms of psychogenic erectile dysfunction is treated with an effervescent formulation according to Example 1 which has been made into a tablet.

The patient is supplied with an effervescent formulation containing 2 mg of apomorphine in the form of a 100 mg tablet. The quantity of apomorphine used is dependent on the severity of the condition and the tolerance of the patient to apormorphine.

The patient places the tablet on the tongue. The tablet effervesces and delivers the apomorphine to the patient.

### EXAMPLE 6

### Treatment of Female Sexual Dysfunction with Apomorphine.

A female patient presenting symptoms of sexual dysfunction is treated with an effervescent formulation according Example 2 which has been made into a tablet.

The patient is supplied with an effervescent formulation containing 3 mg of apomorphine in the form of a 300 mg tablet. The patient places the tablet in the mouth. The tablet effervesces and delivers the apomorphine to the patient.

1. An effervescent formulation comprising apomorphine.

2. An effervescent formulation according to claim 1 comprising multilayer effervescent microspheres.

3. An effervescent formulation according to claim 2 wherein the multilayer effervescent microspheres contain an acidic substance, a basic substance and water-soluble isolating agent.

4. An effervescent formulation according to claim 3 wherein dissolution in water of the multilayer effervescent microspheres leads, after almost immediate effervescence, to a solution or a homogeneous dispersion of the apomorphine.

5. An effervescent formulation according to claim 4 wherein the water-soluble isolating agent is dispersed in the entire bulk of each microsphere, the latter having a two-layer structure: a layer of acidic substance in which is dispersed the water-soluble isolating agent and a layer of alkaline substance in which is dispersed the water-soluble isolating agent.

6. An effervescent formulation according to claim 4 wherein the water-soluble isolating agent is in the form of a thin film separating the acidic and alkaline substances such that each microsphere has a three-layer structure: a layer of acidic substance and a layer of alkaline substance separated by a layer of water-soluble isolating agent.

7. An effervescent formulation according to claim 1 wherein the apomorphine is present in a unit dose amount of from 0.5 mg to 50 mg.

8. An effervescent formulation according to claim 7 wherein the apomorphine is present in a unit dose amount of 2 mg to 3 mg.

9. An effervescent formulation according to claim 1 wherein the formulation is presented in a tablet form.

10. An effervescent formulation according to claim 1 wherein the formulation is presented in a powder form.

11. An effervescent formulation according to claim 1 wherein the apomorphine is present within a microsphere.

12. An effervescent formulation according to claim 1 wherein the apomorphine is not present within a microsphere.

13-14. (canceled)

15. A pharmaceutical composition comprising an effervescent formulation according to claim 1 and a pharmaceutically acceptable carrier.

16. A process for making an effervescent formulation containing apomorphine.

17. A process according to claim 16 wherein the effervescent formulation comprises multilayer effervescent microspheres containing an acidic substance, a basic substance, and a water-soluble isolating agent which upon dissolution in water leads, after almost immediate effervescence, to a solution or a homogeneous dispersion of apomorphine.

18. A process according to claim 17 wherein the acidic and/or basic substances contains or contain apomorphine.

19. A process according to claim 16 further comprising:
forming microspheres, wherein the apomorphine is not present in the microspheres.

20. A process according to claim 18 which employs the method of rotary granulation in a fluidized air bed.

21. A process according to claim 17 wherein basic substance also contains an edible diluant and/or flavourings and/or sweeteners.

22. A process according to claim 17 wherein the apomorphine is present in an amount to give from 0.5 mg to 50 mg in the final unit dosage form.

23. A process according to claim 22 wherein the apomorphine is present in an amount to give from 2 mg to 3 mg in the final unit dosage form.

24. A process according to any one of claim 19 further comprising preparing the microspheres into a tablet.

25. A process according to claim 24 wherein the apomorphine is present on or between the microspheres in the tablet.

26. An effervescent formulation of apomorphine obtained or obtainable by the process of claim 16.

27. A method of treating human male or female sexual dysfunction comprising administering to said human an effervescent formulation of apomorphine according to claim 1.

28. (canceled)

29. The method according to claim 27, wherein the effervescent formulation is present in a pharmaceutical composition that includes a pharmaceutically acceptable carrier.
